(19) United States
(12) Patent Application Publication
(10) Pub. No.: US 2009/0018085 A1
(43) Pub. Date: Jan. 15, 2009

(54) Use of a GnRH antagonist peptide in the treatment of sex hormone-dependent diseases

(75) Inventors: Martin Luck, Hellerup (DK); Pierre Broqua, Southampton (GB)

Correspondence Address: FOLEY AND LARDNER LLP;SUITE 500, 3000 K STREET NW, WASHINGTON, DC, 20007, US

(73) Assignee: Ferring B.V.

(21) Appl. No.: 12/155,897

(22) Filed: Jun. 11, 2008

Related U.S. Application Data
(63) Continuation of application No. 10/380,623, filed on Mar. 17, 2003, now abandoned.
Application No. 10/380,623 is a continuation-in-part of application No. PCT/GB02/03116, filed on Jul. 8, 2002.

(30) Foreign Application Priority Data
Jul. 12, 2001 (GB) 0117057.0

(51) Int. Cl. A61K 38/08; A61P 5/24

(52) U.S. Cl. 514/15

(57) ABSTRACT

A method for treatment of benign prostate hyperplasia, prostate cancer, estrogen-dependent breast cancer, endometrial cancer, ovarian cancer, endometriosis and precocious puberty, or for use for contraceptive purposes or in an in vitro fertilization programme, or for treatment of sex offenders is provided. The method comprises the administration by subcutaneous or intramuscular injection of a therapeutically effective amount of an injectable pharmaceutical composition comprising a solution of a GnRH antagonist peptide according to general formula 1 or a pharmaceutically acceptable salt thereof in a concentration of 0.3-120 mg/ml. Also a pharmaceutical composition and a pharmaceutical kit of parts are provided.

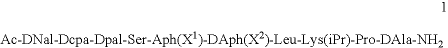

### CONTINUATION APPLICATION DATA

This application is a Continuation-in-Part Application of International Application No. PCT/GB2002/003116 filed in English on Jul. 8, 2002 which was published in English on Jan. 23, 2003 as WO 03/006049, and which claims benefit of British Application No. GB 0117057.0 filed on Jul. 12, 2001. These applications are incorporated herein by reference in their entirety for all purposes.

### FIELD OF THE INVENTION

The present invention relates to the use of a GnRH antagonist peptide in the treatment of sex hormone-dependent diseases.

### BACKGROUND

The discovery and characterization of GnRH (gonadotropin releasing hormone, previously luteinizing hormone releasing hormone, LHRH) as the first mediator in the hypothalamic-pituitary-gonadal axis has opened up new possibilities for the treatment of sex hormone-dependent conditions such as prostate cancer and precocious puberty. A first generation of therapeutic agents were the GnRH superagonists. These acted by continuously stimulating the GnRH receptor, leading to desensitization of the pathway. However, these agents tend to provoke a “flare” reaction and so are being displaced by a second generation, the GnRH antagonists.

A problem arises due to the need for chronic administration of the therapeutic agents. Like the superagonists before them, the current generation of GnRH antagonists are peptides that are unsuited for oral administration. Subcutaneous or intramuscular injection works well with the compounds, but daily injections would not be acceptable to the patient population and so current research is aimed at developing depot formulations of the antagonists. For the superagonists such depot technology is well established. The peptide is released from a biodegradable polymer matrix over a period of (typically) one to three months. The transfer of this technology to the antagonists is complicated by the need to administer larger quantities of drug substance. As a result, there has been a significant effort made to develop antagonists that are more potent (so requiring less drug substance to be included in the depot) or that have physicochemical properties compatible with higher drug/polymer ratios, as well as efforts directed to the development of more sophisticated depot technologies.

U.S. Pat. No. 5,925,730 (corresponding to International Patent Application PCT/US98/07438, EP 1 003 774) discloses, inter alia, GnRH antagonist peptides according to general formula 1.

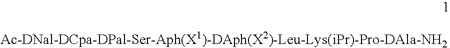

These peptides have a high affinity for the GnRH receptor and are much more soluble in water than previously described GnRH analogues. It was suggested in the disclosure that the increased solubility of these compounds is, at least in part, responsible for the long duration of action of up to three or four days in some in vivo models. It has also been suggested that the duration of action of these compounds is dose-related, i.e. that the duration of action is dependent on the amount of peptide given. However, the optimum conditions for formulating these peptides were not discussed.

### SUMMARY OF THE INVENTION

We have now discovered that certain peptides according to general formula 1 are capable of forming a gel after subcutaneous injection, and that this gel can act as a depot from which the peptide is released over a period of weeks or even months. We have also found that the key variable is the concentration of the solution rather than the amount of substance administered. The concentration of the solution must be within a certain range. If the solution is too dilute then no depot is formed and the long duration of action is lost, no matter how much drug substance is given. If the solution is too concentrated then gel formation will occur before the drug can be administered.

Thus, according to a first aspect, the present invention relates to a method for treatment of certain disorders of the genitourinary tract and other sex-hormone dependent conditions, wherein an injectable pharmaceutical composition comprising a solution in a pharmaceutically acceptable solvent of a GnRH antagonist peptide is administered by subcutaneous or intramuscular injection of a therapeutically effective amount of said pharmaceutical composition, wherein the concentration of the GnRH antagonist peptide in the solution is not less than 0.3 mg/ml and not more than 120 mg/ml. Preferably, the method provides for the continuous release of a GnRH antagonist peptide over a period of more than two weeks.

Furthermore, according to a second aspect, the present invention relates to an injectable pharmaceutical composition comprising a solution in a pharmaceutically acceptable solvent of a GnRH antagonist peptide or a pharmaceutically acceptable salt thereof.

The composition may be presented in a form that is ready for administration, but is preferably presented as a kit of parts comprising the peptide as a solid and solvent, such that the solution can be made up immediately prior to administration. Thus, according to a third aspect, the present invention relates to a pharmaceutical kit of parts comprising a first component which comprises a GnRH antagonist peptide or a pharmaceutically acceptable salt thereof and a second component which comprises a pharmaceutically acceptable solvent suitable for said GnRH antagonist peptide or a pharmaceutically acceptable salt thereof. The kit is provided such that the components upon use can be mixed to provide an injectable pharmaceutical composition having a concentration of the peptide in the solution that is not less than 0.3 mg/ml and not more than 120 mg/ml.

As stated above, the method is suitable for treatment of certain disorders of the genitourinary tract and other sex-hormone dependent conditions. The injectable pharmaceutical composition and the pharmaceutical kit of parts are also suitable for treatment of these disorders.

More precisely, the method, the pharmaceutical composition and the pharmaceutical kit are suitable for treatment of a condition selected from the group consisting of benign prostate hyperplasia, prostate cancer, estrogen-dependent breast cancer, endometrial cancer, ovarian cancer, endometriosis and precocious puberty.

The method, the pharmaceutical composition and the pharmaceutical kit are also suitable for the use for contraceptive purposes.

Furthermore, the method, the pharmaceutical composition and the pharmaceutical kit are suitable for the treatment of sex offenders since they provide means for chemical castration.

The GnRH antagonist peptide used in the method, the pharmaceutical composition or the pharmaceutical kit is a peptide having general formula 1

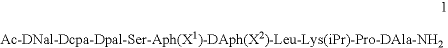

wherein X¹ and X² are selected independently from L- and D-Hor, L- and D-Imz and CONHR, and wherein R is hydrogen or a lower alkyl, i.e. an alkyl comprising 1-6 carbon atoms.

### DETAILED DESCRIPTION OF THE INVENTION

The present invention is based on the use of a GnRH antagonist peptide according to general formula 1, or a pharmaceutically acceptable salt thereof.

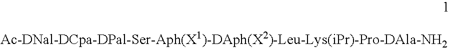

In this general formula 1 the abbreviations have the following meanings:
Ac Acetyl
DNal D-β-(2-naphthyl)alanine
DCpa D-4-chlorophenylalanine
DPal D-β-(3-pyridyl)alanine
Ser Serine
Aph(X¹) 4-aminophenylalanine wherein the ω-amino group has a substituent X¹
DAph(X²) D-4-aminophenylalanine wherein the ω-amino group has a substituent X²
Leu Leucine
Lys(iPr)- N^{ω}-isopropyllysine
Pro Proline
DAla-NH₂ D-alanine amide

The substituents X¹ and X² are independently selected from carbamoyl groups —CONHR, where R is H or a lower (C₁-C₆) alkyl group, D- and L-hydroorotyl (D- and L-Hor) groups, and D- and L-2-imidazolidone-4-carbonyl (D- and L-Imz) groups.

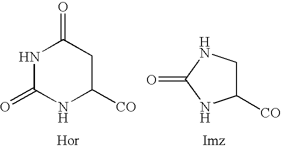

In a preferred embodiment of the invention X¹ is D- or L-Hor. In another preferred embodiment of the invention X² is a carbamoyl group. In a more preferred embodiment of the invention X¹ is D- or L-Hor and X2 is a carbamoyl group. In a most preferred embodiment of the invention X¹ is L-Hor and X² is a carbamoyl group CONH₂.

Peptides according to the above definition are capable of forming salts. In particular, they are capable of forming addition salts with acids such as hydrochloric acid, acetic acid and trifluoroacetic acid. Provided that they are pharmaceutically acceptable, all such salts are included within the scope of the present disclosure. The acetate and hydrochloride salts are particularly preferred according to the present invention.

The method according to the invention may be used for treatment of a patient in need of such treatment, i.e. any human or non-human mammal suffering from at least one condition selected treatable according to the invention, as further explained below. The composition used according to the present invention releases the GnRH antagonist peptide into the general circulation over a period of several days, weeks, or even months. Accordingly, it causes long term blockade of the GnRH receptor, which results in a profound suppression of the release of LH and FSH. This in turn results in a suppression of gonadal function, including suppression of the release of sex steroid hormones from the gonads. Hence the compositions according to the present invention are useful in the treatment of diseases which involve stimulation of a tissue by sex steroid hormones or directly by LH or FSH. Such diseases include benign prostate hyperplasia, prostate cancer, estrogen-dependent breast cancer, endometrial cancer, ovarian cancer, endometriosis and precocious puberty. The term “treatment” used herein relates to both treatment in order to cure or alleviate at least one of these conditions, and to treatment in order to prevent the development of at least one of these conditions. The treatment may either be performed in an acute or in a chronic way. The compositions may also be used as contraceptive agents, particularly male contraceptive agents. When used for this purpose it may be necessary to administer testosterone in order to maintain libido. Further uses for the compositions include the regulation of ovarian function in the context of an in vitro fertilization programme and as behavior-modifying agents for the treatment of sex offenders.

The method of treatment according to the present invention may be used as the sole treatment for the disease. Alternatively, the attending physician may choose to combine the method with other treatments given simultaneously or serially. Other treatments may include the administration of other pharmaceutical agents, including those acting by mechanisms independent of the GnRH-LH/FSH-gonad pathway, and non-pharmaceutical treatments such as surgery.

The peptide or pharmaceutically acceptable salt is preferably administered by injection, more preferably by subcutaneous injection or intramuscular injection. For these purposes, the peptide or pharmaceutically acceptable salt is preferably comprised in a solution in a pharmaceutically acceptable solvent. Examples of such pharmaceutically acceptable solvents are water, an alcohol (for example ethanol), N-methylpyrrolidone or dimethylsulfoxide. In a preferred embodiment of the invention the solvent is water or a mixture of water and a second solvent, such as alcohol, N-methylpyrrolidone or dimethylsulfoxide, such that the water constitutes at least 90% by weight of the solvent mixture. The composition comprising the peptide or pharmaceutically acceptable salt and the pharmaceutically acceptable solvent may contain other components such as osmotic pressure regulating agents, for example sodium chloride and mannitol, preservatives, buffering agents and the like. In a preferred embodiment of the invention, the concentration of sodium chloride is below 2 mg/ml. In a more preferred embodiment, sodium chloride is absent from the composition and mannitol is used to adjust the osmolarity of the solution. The composition may further include additional pharmaceutically active agents, but it is preferred that the said GnRH antagonist peptide should be the only such agent.

The composition according to the present invention may be presented in a form that is ready for immediate use, such as a solution in a sealed container or a prefilled syringe. Alternatively and preferably, the composition may be presented in a form that requires some preparation prior to administration. For example, the composition may be presented as a kit of parts, including a sealed container containing the peptide as a lyophilized powder and a second container containing the solvent. The peptide may be freeze dried. Further components may be included with the solid or liquid part. Thus the kit may comprise a first container containing the peptide and a second containing isotonic saline, or a first container containing the peptide and mannitol and a second container containing sterile water. Prior to administration the solvent is added to the container containing solid component in order to give the solution for injection.

An essential property of the present invention is that the solution used is stable prior to administration but converts into a gel immediately after administration. This property is a function of the concentration of the peptide.

The precise concentration range effective for the purposes of the invention may vary somewhat from case to case, e.g. according to the identities of peptide and solvent and of secondary ingredient(s) when present, and to intended storage time. It is evident that in any given instance the result to be achieved and the effective concentration range therefor are directly and positively verifiable by the simplest tests and observations requiring minimal experimentation. As a general guide, a minimum peptide concentration of 0.3 mg/ml should be sufficient for injection to result in gel formation at the injection site at a rate and to an extent which are satisfactory.

When the composition is to be presented as or stored as a made-up solution the peptide concentration will usually be not more than 5 mg/ml to prevent gel formation during storage (e.g. for up to 4 weeks), and not less than 0.3 mg/ml to ensure that the gel forms soon after administration.

When the composition is presented as a kit of parts to be administered immediately after mixing (e.g. within 30 minutes of mixing), the peptide concentration in the final solution may be higher, for example as much as 120 mg/ml.

The minimum concentration is not dependent on the way in which the composition is presented, since it is determined by the need to form a gel after injection.

In a preferred embodiment of the present invention the concentration of the peptide is not more than 80 mg/ml. In a more preferred embodiment, the concentration of the peptide is not more than 40 mg/ml. In another preferred embodiment of the present invention the concentration of the peptide is not less than 1 mg/ml. In another more preferred embodiment, the concentration of the peptide is not less than 5 mg/ml. In a still further preferred embodiment the concentration of the peptide is between 5 mg/ml and 80 mg/ml, such as not less than 5 mg/ml and not more than 40 mg/ml.

In a still further embodiment the concentration of the peptide is between 5 mg/ml and 80 mg/ml. Peptide at concentrations within this range (for example 20 mg/ml, or 25 mg/ml) may be used to form a gel after administration which releases the peptide over a period of at least two weeks, preferably for a period of three months.

In general the attending physician will decide on the details of the dosology by taking into consideration the desired therapeutic outcome and the medical history and current condition of the patient. The volume of composition administered will generally be from 1 to 10 ml, giving for example a peptide dose of 0.3 to 1200 mg. Administration will be by subcutaneous or intramuscular injection, preferably by subcutaneous injection, at a single site or divided between two or more sites. The administration will be repeated at appropriate intervals of two weeks to three months for the duration of the treatment.

The present invention is illustrated further in the following non-limiting Examples.

### EXAMPLES

### Example 1

### Preparation of Peptides

The peptides used in the compositions of the present invention can be prepared according to the methods described in U.S. Pat. No. 5,925,730. In particular, the peptide Ac-DNal-DCpa-DPal-Ser-Aph(L-Hor)-DAph(CONH₂)-Leu-Lys(iPr)-Pro-DAla-NH₂ (“Peptide 1”) was prepared according to the method of Example 1 of the US patent and isolated as its acetate salt.

### Example 2

### Stability of Aqueous Solution

Peptide 1 was dissolved in water at various concentrations, and the resulting solutions were allowed to stand at room temperature for an extended period of time. Gel formation was determined by visual examination. The observations are summarized in Table 1.

TABLE 1
Stability of aqueous solutions

| Concentration* | |
|---|---|
| (mg/ml) | Stability |
| 0.25 | No gel formation after 6 months |
| 1.0 | No gel formation after 6 months |
| 5.0 | Gel formation after 4 weeks |
| 10.0 | Gel formation after 2 weeks |
| 30.0 | Gel formation after 48 hours |
| 40.0 | Gel formation after 24 hours |
| 60.0 | Gel formation after 8 hours |
| 80.0 | Rapid gel formation within 60 minutes |
| 120.0 | Rapid gel formation within 30 minutes |

*calculated as free base

### Example 3

### Minimum Concentration Needed to Form Gel in Vivo

Peptide 1 was dissolved in 5% mannitol at various concentrations and injected subcutaneously into rats. The animals were sacrificed after 24 hours and the injection site was dissected and examined. When deposits of gel were found these were removed and weighed to assess completeness of gel formation. Significant gel formation was observed with concentrations of peptide greater than 0.3 mg/ml.

### Example 4

### Efficacy of Formulation in Vivo

Peptide 1 is dissolved in 5% mannitol (25 mg/ml). Three ovariectomised Rhesus monkeys are treated with this solution (80 μl/kg) by subcutaneous injection. Serum LH levels is measured for the following 101 days. The results are summarized in Table 2.

TABLE 2
Biological action

| Time | Serum LH (ng/ml), mean ± se |
|---|---|
| 0 | 60.1 ± 7.5 |
| Hour 6 | 16.2 ± 1.9 |
| Day 1 | 10.5 ± 1.5 |
| Day 2 | 11.8 ± 2.6 |
| Day 7 | 6.7 ± 1.2 |
| Day 14 | 5.8 ± 0.9 |
| Day 21 | 6.6 ± 1.0 |
| Day 28 | 9.4 ± 1.3 |
| Day 35 | 8.8 ± 1.0 |
| Day 42 | 11.8 ± 2.3 |
| Day 72 | 29.5 ± 4.3 |
| Day 101 | 48.9 ± 8.3 |

### Example 5

### Efficacy of Formulation in Vivo

Peptide 1 is dissolved in 5% mannitol (5 mg/ml). Three adult male Beagle dogs are treated with this solution (100 μl/kg) by subcutaneous injection. Serum Testosterone levels are measured for the following 42 days. When Testosterone levels started to incline again the experiment was terminated. The results are summarized in Table 3.

TABLE 3
Biological action

| | Serum Testosterone (nmol/L), |
|---|---|
| Time | mean ± se |
| 0 | 13.8 ± 5.7 |
| Hour 4 | <0.2 |
| Day 7 | <0.2 |
| Day 14 | <0.2 |
| Day 21 | <0.2 |
| Day 28 | <0.2 |
| Day 42 | 3.3 ± 3 |

### Example 6

### Compositions According to the Invention

### 6A—Solution for Injection

A solution is prepared by dissolving 51.84 g of the peptide Ac-DNal-DCpa-DPal-Ser-Aph(L-Hor)-DAph(CONH₂)-Leu-Lys(iPr)-Pro-DAla-NH₂ acetate Peptide 1, see Example 1) and 500 g of mannitol in 10 liters of sterile water to give a final concentration of 5 mg/mi of peptide (calculated as the free base) in 5% aqueous mannitol. The solution is filtered through a 0.2-micron filter and divided into 5000 glass vials to provide 5000 individual doses of the solution, each of 2 ml.

### 6B—Two-Component Kit

A solution is prepared by dissolving 414.7 g of the peptide Ac-DNal-DCpa-DPal-Ser-Aph(L-Hor)-DAph(CONH₂)-Leu-Lys(iPr)-Pro-DAla-NH₂ acetate (Peptide 1, see Example 1) and 250 g of mannitol in 10 liters of sterile water. The solution is filtered through a 0.2-micron filter and divided into 5000 glass vials, then frozen and lyophilized.

A second solution is prepared by dissolving 250 g of mannitol in 10 liters of sterile water. This solution is filtered through a 0.2-micron filter and divided into 5000 glass vials. A kit is then made up of one vial of lyophilisate and one of mannitol solution, such that when the lyophilisate is dissolved in the mannitol solution prior to administration there results a 2 ml dose of a 40 mg/ml solution of the peptide in 5% aqueous mannitol.

The data presented in Example 2 establishes a maximum concentration above which the peptides form gels too rapidly to be conveniently administered in a clinical situation. Example 3 establishes a minimum concentration below which the peptides do not form gels following administration and so would not give the desired long duration of action. Examples 4 and 5 demonstrate that the compositions according to the present invention are effective in blocking the release of LH and testosterone in an animal model. Such results are widely acceptable as an indicator of clinical efficacy in human steroid dependent pathologies. Hence they are illustrative of the clinical utility of the compositions of the invention such as, but not limited to, those of Example 6.

1-70. (canceled)

71. A method for treatment of a condition selected from the group consisting of benign prostate hyperplasia, prostate cancer, estrogen-dependent breast cancer, endometriosis and precocious puberty, or for use for contraceptive purposes, or for use in an in vitro fertilization programme, or for treatment of sex offenders, which method comprises the administration by subcutaneous or intramuscular injection of a therapeutically effective amount of an injectable pharmaceutical composition comprising a solution in a pharmaceutically acceptable solvent of a GnRH antagonist peptide according to formula 1 or a pharmaceutically acceptable salt thereof 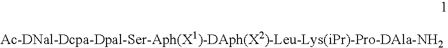
wherein X¹ is L-Hor and X² is CONH₂,
wherein the concentration of the GNRH antagonist peptide or salt thereof in the solution is selected from 5, 10, 20, 25, 40, 60, 80 and 120 mg/ml.

72. The method of claim 71, wherein the solvent is selected from the group consisting of (i) water and (ii) a mixture of water and a second solvent such that at least 90% by weight of the solvent is water.

73. The method of claim 71, wherein the concentration of the said peptide or salt thereof is such that the peptide or salt thereof spontaneously forms a gel after administration and said gel acts as a depot which releases the peptide or salt thereof over a period of at least two weeks.

74. The method of claim 71, wherein the concentration of the said peptide or salt thereof is such that the peptide or salt thereof spontaneously forms a gel after administration and the gel acts as a depot which releases the peptide or salt thereof over a period of at least three months.

75. The method of claim 71, wherein the composition comprises hydrochloride or acetate salt of the peptide.

76. An injectable pharmaceutical composition comprising a solution in a pharmaceutically acceptable solvent of a GNRH antagonist peptide according to formula 1 or a pharmaceutically acceptable salt thereof 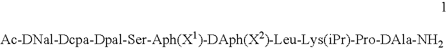
wherein X¹ is L-Hor and X² is CONH₂,
wherein the concentration of the peptide or salt thereof in the solution is selected from 5, 10, 20, 25, 40, 60, 80 and 120 mg/ml.

77. The injectable pharmaceutical composition of claim 76, wherein the solvent is selected from the group consisting of (i) water and (ii) a mixture of water and a second solvent such that at least 90% by weight of the solvent is water.

78. The injectable pharmaceutical composition of claim 76, wherein the concentration of the said peptide or salt thereof is such that the peptide or salt thereof spontaneously forms a gel after administration and said gel acts as a depot which releases the peptide or salt thereof over a period of at least two weeks.

79. The injectable pharmaceutical composition of claim 76, wherein the concentration of the said peptide or salt thereof is such that the peptide or salt thereof spontaneously forms a gel after administration and the gel acts as a depot which releases the peptide or salt thereof over a period of at least three months.

80. The injectable pharmaceutical composition of claim 76, wherein the composition comprises the hydrochloride or acetate salt of the peptide.

81. A pharmaceutical kit of parts comprising
(A) a first component, comprises of a GnRH antagonist peptide according to formula 1 or a pharmaceutically acceptable salt thereof 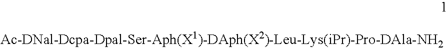
wherein X¹ is L-Hor and X² is CONH₂, and
(B) a second component, comprised of a pharmaceutically acceptable solvent therefor,
such that said components can be mixed to provide an injectable pharmaceutical composition having a concentration of the peptide or salt thereof in the solution that is selected from 5, 10, 20, 25, 40, 60, 80 and 120 mg/ml.

82. The pharmaceutical kit of claim 81, wherein the solvent is selected from the group consisting of (i) water and (ii) a mixture of water and a second solvent such that at least 90% by weight of the solvent is water.

83. The pharmaceutical kit of claim 81, wherein the concentration of the said peptide or salt thereof is such that the peptide or salt thereof spontaneously forms a gel after administration and said gel acts as a depot which releases the peptide or salt thereof over a period of at least two weeks.

84. The pharmaceutical kit of claim 81, wherein the concentration of the said peptide or salt thereof is such that the peptide or salt thereof spontaneously forms a gel after administration and the gel acts as a depot which releases the peptide or salt thereof over a period of at least three months.

85. The pharmaceutical kit of claim 81, wherein the first component comprises the hydrochloride or acetate salt of the peptide.

86. The pharmaceutical kit of claim 81, further comprising instructions for using the kit in a method for the treatment of a condition selected from the group consisting of benign prostate hyperplasia, prostate cancer, estrogen-dependent breast cancer, endometriosis and precocious puberty, or for use for contraceptive purposes, or for use in an in vitro fertilization programme, or for treatment of sex offenders.

87. A method for treatment of a condition selected from the group consisting of benign prostate hyperplasia, prostate cancer, estrogen-dependent breast cancer, endometriosis and precocious puberty, or for use for contraceptive purposes, or for use in an in vitro fertilization programme, or for treatment of sex offenders, which method comprises the administration by subcutaneous or intramuscular injection of a therapeutically effective amount of an injectable pharmaceutical composition comprising a solution in a pharmaceutically acceptable solvent of a GNRH antagonist peptide according to formula 1 or a pharmaceutically acceptable salt thereof 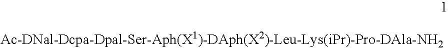
wherein X¹ is L-Hor and X² is CONH₂,
wherein the concentration of the GnRH antagonist peptide or salt thereof in the solution is from 5 to 25 mg/ml or from 60 to 120 mg/ml.

88. An injectable pharmaceutical composition comprising a solution in a pharmaceutically acceptable solvent of a GNRH antagonist peptide according to formula 1 or a pharmaceutically acceptable salt thereof 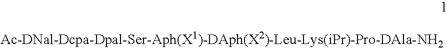
wherein X¹ is L-Hor and X² is CONH₂,
wherein the concentration of the peptide or salt thereof in the solution is from 5 to 25 mg/ml or from 60 to 120 mg/ml.

89. A pharmaceutical kit of parts comprising
a first component, which comprises a GnRH antagonist peptide according to formula 1 or a pharmaceutically acceptable salt thereof 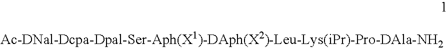
wherein X¹ is L-Hor and X² is CONH₂, and
a second component, which comprises a pharmaceutically acceptable solvent therefor,
such that said components can be mixed to provide an injectable pharmaceutical composition having a concentration of the peptide or salt thereof in the solution that is from 5 to 25 mg/ml or from 60 to 120 mg/ml.


SEQUENCE LISTING (1 sequence)

SEQ ID NO: 1 - protein, 10 aa, Artificial sequence
  Synthetic peptide.
XXXSXXLKPA